(19) United States
(12) Patent Application Publication
(10) Pub. No.: US 2026/0009040 A1
(43) Pub. Date: Jan. 8, 2026

(54) FUSION POLYPEPTIDES FOR CELL PENETRATION AND CELL TARGETING

(71) Applicant: Christoph KUTZNER, Mannheim (DE)

(72) Inventor: Christoph KUTZNER, Mannheim (DE)

(21) Appl. No.: 18/881,680

(22) PCT Filed: Jul. 6, 2023
(86) PCT No.: PCT/EP2023/068719

§ 371 Date: Jan. 6, 2025

(30) Foreign Application Priority Data
Jul. 7, 2022 (EP) 22183641.4

(51) Int. Cl. C12N 15/62; C12N 15/70; C12N 15/82

(52) U.S. Cl. CPC C12N 15/625; C12N 15/70; C12N 15/8202; C07K 2319/02; C07K 2319/50

(57) ABSTRACT

The present invention concerns fusion polypeptides comprising a cell penetration and a substrate binding domain. Additionally, the present invention concerns a genetically modified cell expressing these fusion polypeptides as well as the use of these fusion polypeptides for introducing a substance into a target cell.

The present invention concerns fusion polypeptides comprising a cell penetration and a substrate binding domain. Additionally, the present invention concerns a genetically modified cell expressing these fusion polypeptides as well as the use of these fusion polypeptides for introducing a substance into a target cell.

Delivering substances to the cell is the basis for e.g. a successful drug-targeting or specific epigenetic modification. For this purpose active pharmaceutical ingredients or DNA/RNA sequences must be introduced into a target cell. To achieve an introduction of a sequence into a cell, the cell barrier must be passed, which protects the cell against environmental influences. Besides the protection from environmental influences, the cell requires certain substances and signal molecules from other cells of the organism, which need to have access to the cell.

There are several mechanism for mass transport in and out of a cell for retaining the natural balance and provide the required substances to a cell. The independent and undirected mass transport ways into the cell are diffusion and osmosis. These processes do not require energy and are thus called passive mass transport. Active mass transport requires energy. Such active transport processes are e.g. membrane proteins, endocytosis and exocytosis.

The natural transport processes inside the cell can be targeted to deliver active substances, such as e.g. pharmaceuticals to a cancer cell or genetic information for gene silencing.

Another way of introducing substances into a cell is the use of cell-penetrating peptides (CPP). These peptides allow an energy-independent pathway for delivering substances inside a cell, which were thought to be only deliverable by nearly energy-dependent pathways. CPPs are associated with the substances, which are thought to be introduced into a cell (“cargo”), either through covalent bonds or non-covalent interactions. CPPs can be of different sizes, amino acid sequences, and charges, but all CPPs have the ability to translocate the plasma membrane and facilitate the delivery of various molecular cargoes to the cytoplasm or an organelle.

CPPs are specific for certain organisms or cell-types depending on the cell membrane composition. Plant cells require different CPPs than mammalian cells for introducing the cargo into the cell. Numata et al. (“Library screening of cell-penetrating peptide for BY-2 cells, leaves of Arabidopsis, tobacco, tomato, poplar and rice callus”, (2018)8:10966, Nature scientific reports) report the screening of different CPPs for targeting different plant species. It was immanent that the results for the different CPPs vary strongly between the different target plants and are dependent on the secondary structure, the CPPs are forming.

One big challenge in utilizing CPPs is the dependency of the transport efficiency on the cargo. It would be preferred if CPPs that were identified to have a good transport efficiency can be used for a wide variety of different cargo, e.g. transporting polypeptides as well as small molecules or DNA/RNA.

CPPs, especially cationic peptides, are assumed to enable the membrane passing of fusion polypeptides by non-endosomal mechanisms. The CPPs Tat and Arg9 were compared to find their ability to transduce polypeptides but also nucleic acids and it was found that a large portion of the CPP labelled cargo was simply adhered to the cell surface and not transported into cell, while nucleic acid cargo was introduced (“Cell-penetrating peptides-A Reevaluation Of The Mechanism Of Cellular Uptake”, Richard et al., The Journal of biological Chemistry, 278(1) (585-590), 2003).

It is thus desirable to obtain CPPs, which can be used for different types of cargo without screening for each type of cell and each type of cargo the optimal.

Another drawback in the utilization of CPPs for cargo delivery into cells is that cell penetrating peptides as transport molecules are difficult to be produced in the amounts for achieving the concentrations that are needed for delivering an appropriate amount of cargo into the cell. Furthermore, the applications for CPP-mediated transport are limited due to high molecular weight and high amount of required negative charges. The uptake of cargo via CPP-mediated transport is low compared to the high amount of peptide needed. Fusion polypeptides could be a way to overcome this low efficiency problem, but covalent linkage of the cargo could lead to an inefficient effect of the substance in the cell.

Thus, the primary object of the present invention was to provide a tool for targeted delivery of cargo inside a cell, which is applicable for a variety of different cell types in different organisms and can be used with different types of cargo.

Another object of the present invention was to provide such tool, which can be produced economically and easy to scale-up.

The primary object of the present invention was solved by providing a fusion polypeptide comprising or consisting of at least three functional domains:
i) a substrate binding domain binding to a substrate non-covalently, wherein the substrate is selected from the group consisting of polypeptides, polysaccharides, DNA, RNA or a mixture of DNA and RNA;
ii) a cell penetrating domain;
iii) a signal sequence;
iv) optionally a linker domain; and
v) optionally a pH-sensitive autoprotease domain,
   wherein the cell penetrating domain has a length of between 15 and 50 amino acid residues, preferably of 20 to 40 amino acid residues and especially preferably of between 24 to 34 amino acid residues, and
   wherein the cell penetrating domain comprises at least 70%, preferably at least 75% and especially preferably 80% amino acid residues with a helix potential value >1, dependent on the overall length of the cell penetrating domain.

The substrate binding domain of the fusion polypeptide according to the invention receives the substrate or cargo, which should be introduced into the cell. The fusion polypeptide according to the invention is brought into contact with the cargo and the cargo binds non-covalently to the substrate binding domain.

It was surprisingly found in terms of the present invention that the combination of the substrate binding domain and the cell penetration domain allows the complex of fusion polypeptide and substrate to permeate the cell membrane. The type of nucleic acid is irrelevant to the binding and transport process. Naturally occurring DNA or RNA binding domains are restricted to binding a specific substrate. The substrate binding domains of the present invention were able to bind a high variety of different DNA and/or RNA sequences independent of compositions, length of sequences, circular or linear sequences or DNA and RNA. It was surprising that the substrate binding domain binds to DNA and RNA as the natural occurring CBMs of C. chromiireducens of which the substrate binding domains are based, exhibit their function at pH values, where DNA and RNA are not stable. The substrate binding domains of the present invention are able to bind DNA and RNA at pH values ensuring the stability of the nucleic acid molecules.

Another advantage of the substrate binding domain in comparison to other reported DNA/RNA binding domains is that the sequence of the DNA/RNA molecule is not altered.

It was discovered that besides the receiving of the cargo, which should be introduced into the cell, the substrate binding domain plays also a role in target delivery of the cargo inside the cell, as it can also bind to cellular structures such as DNA/RNA or the actin cytoskeleton. When cellular structures consisting of polysaccharides or exhibiting polysaccharide motifs are present, the substrate binding domain has a higher affinity to such substrates and releases the DNA/RNA cargo inside the cell.

The cell penetrating domain of the fusion polypeptide according to the invention allows the cargo to be introduced into the cell by triggering the membrane translocation mechanism. Preferably, the cell penetrating domain of the fusion polypeptide according to the invention triggers the endocytosis-mediated translocation or translocation through the formation of a transitory structure.

It was surprisingly found that the use of a cell penetrating domain having a length of between 15 and 50 amino acid residues, preferably of 20 to 40 amino acid residues and especially preferably of between 24 to 34 amino acid residues, and comprising at least 70%, preferably at least 75% and especially preferably 80% amino acid residues with a helix potential value >1, dependent on the overall length of the cell penetrating domain, allow a high efficiency of introduced cargo into the cell as well as the compatibility with a variety of different organisms.

The helix potential (Pα) is the determined value for the frequency of amino acids to be incorporated into α-helical secondary structures. Amino acids having a Pα value of >1 are helical forming amino acids, whereas amino acids having a Pα value of <1 are helical disrupting amino acids. Table 1 shows an overview of the helix potentials of proteinogenic amino acids according to Stryer Biochemie, 5^{th} edition, page 73, Table 3.3.

The cell penetrating domain according to the invention thus forms a compact stable secondary helical structure, which leads to an overall improved cell penetrating performance.

TABLE 1
Overview of amino acids and their helix potential

| Amino acid | Helix potential (Pα) |
|---|---|
| Glutamic acid (Glu) | 1.59 |
| Alanine (Ala) | 1.41 |
| Leucine (Leu) | 1.34 |
| Methionine (Met) | 1.30 |
| Glutamine (Gln) | 1.27 |
| Lysine (Lys) | 1.23 |
| Arginine (Arg) | 1.21 |
| Phenylalanine (Phe) | 1.16 |
| Isoleucine (Ile) | 1.09 |
| Histidine (His) | 1.05 |
| Tryptophan (Trp) | 1.02 |
| Aspartic acid (Asp) | 0.99 |
| Valine (Val) | 0.90 |
| Threonine (Thr) | 0.76 |
| Asparagine (Asn) | 0.76 |
| Cystein (Cys) | 0.66 |
| Tyrosine (Tyr) | 0.61 |
| Serine (Ser) | 0.57 |
| Glycine (Gly) | 0.43 |
| Proline (Pro) | 0.34 |

The signal sequence of the fusion polypeptide according to the invention allows the fusion polypeptide to be guided to certain cell compartments. This is beneficial either in the production of these fusion polypeptides in recombinant expression systems as well as in the cellular targeting of the cargo bound to the substrate binding domain of the fusion polypeptide.

Preferably, the signal sequence of the fusion polypeptide according to the invention is an inclusion body promoting sequence or a secretion sequence, preferably of secretion type IV or type II of gram negative bacteria. These sequences allow the production of the fusion polypeptide according to the invention in gram-negative recombinant production systems, such as e.g. E.coli.

Moreover preferably, the signal sequence of the fusion polypeptide according to the invention is a commonly known signal sequence, e.g. as described in Stryer Biochemie, 5. Auflage, table 12.4.

Preferably, the present invention relates to a fusion polypeptide according to the invention, wherein the cell penetrating domain comprises or consists of at least one amino acid sequence selected from the group consisting of SEQ ID NOs.: 1 to 24, or an amino acid sequence having a sequence identity of more than 90%, preferably at least 91%, preferably at least 92%, preferably at least 93%, preferably at least 94%, preferably at least 95%, preferably at least 96%, preferably at least 97%, preferably at least 98% preferably at least 99% to an amino acid sequence selected from the group consisting of SEQ ID NOs.: 1 to 24.

Especially preferably, the present invention relates to a fusion polypeptide according to the invention, wherein the cell penetrating domain comprises or consists of at least one amino acid sequence selected from the group consisting of SEQ ID NOs.: 1, 2, 3, 17, 18, 19, 20 and 21or an amino acid sequence having a sequence identity of more than 90%, preferably at least 91%, preferably at least 92%, preferably at least 93%, preferably at least 94%, preferably at least 95%, preferably at least 96%, preferably at least 97%, preferably at least 98% preferably at least 99% to an amino acid sequence selected from the group consisting of SEQ ID NOs.: 1, 2, 3, 17, 18, 19, 20 and 21.

Whenever the present disclosure relates to the percentage of identity of nucleic acid or amino acid sequences to each other these values define those values as obtained by using the EMBOSS Water Pairwise Sequence Alignments (nucleotide) program or the EMBOSS Water Pairwise Sequence Alignments (polypeptide) program for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see Smith, T. F. & Waterman, M. S. “Identification of common molecular subsequences” Journal of Molecular Biology, 1981 147 (1): 195-197). When conducting an alignment, the default parameters defined by the EMBL-EBI are used. Those parameters are (i) for amino acid sequences: Matrix=BLOSUM62, gap open penalty=10 and gap extend penalty =0.5 or (ii) for nucleic acid sequences: Matrix=DNAfull, gap open penalty=10 and gap extend penalty=0.5. The skilled person is well aware of the fact that, for example, a sequence encoding a polypeptide can be “codon-optimized” if the respective sequence is to be used in another organism in comparison to the original organism a molecule originates from.

Preferably, the present invention relates to a fusion polypeptide according to the invention, wherein the substrate binding domain comprises at least one sequence of the amino acid motif sequences IHYWN and VYVKV.

The preferred amino acid motif sequences in terms of the present invention were found to have a significant influence on the binding properties of the substrate binding domain and can be found in all substrate binding domains according to the invention.

Furthermore, it is preferred if two, three, four, five or more repetitions of these two amino acid motif sequences are present in the substrate binding domain according to the invention. Preferably, the repetitions are incorporated in the substrate binding domain in a consecutive order or separated by short sequences with hydrophobic amino acids. It is preferred in terms of the present invention if the short sequences with hydrophobic amino acids have a maximum length of 20 amino acids, wherein at least 50%, preferably 60%, especially preferably 70% amino acids dependent on the short sequence length are hydrophobic amino acids.

Hydrophobic amino acids in terms of the present invention are amino acids selected from the group consisting of alanine, valine, leucine, isoleucine, phenylalanine, proline, tryptophan and methionine.

Preferably, the present invention relates also to a fusion polypeptide according to the present invention, wherein the substrate binding domain comprises or consist of an amino acid sequence having a length of between 200 and 600, preferably of between 250 and 550 amino acids.

Moreover preferably, the present invention relates to a fusion polypeptide according to the invention, wherein the substrate binding domain comprises or consists of at least one amino acid sequence selected from the group consisting of SEQ ID Nos.: 25 and 26 or an amino acid sequence having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to an amino acid sequence selected from the group consisting of SEQ ID Nos.: 25 and 26.

Preferably, the present invention relates to a fusion polypeptide according to the invention, wherein the signal sequence is an inclusion body promoting sequence or a secretion sequence, preferably a secretion sequence of secretion type IV or type II of gram negative bacteria.

Moreover preferably, the present invention relates to a fusion polypeptide according to the invention, wherein the signal sequence comprises or consists of at least one amino acid sequence selected from the group consisting of SEQ ID Nos.: 27 and 28 or an amino acid sequence having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to an amino acid sequence selected from the group consisting of SEQ ID Nos.: 27 and 28.

Preferably, the fusion polypeptide according to the invention comprises a linker domain and preferably, the linker domain comprises an N-terminal alpha helix and/or a C-terminal sequence of a random coil structure, preferably wherein the linker domain comprises or consists of at least one amino acid sequence selected from the group consisting of SEQ ID Nos.: 29 and 30 or an amino acid sequence having at least 90%, preferably at least 91%, preferably at least 92%, preferably at least 93%, preferably at least 94%, preferably at least 95%, preferably at least 96%, preferably at least 97%, preferably at least 98% or preferably at least 99% sequence identity to an amino acid sequence selected from the group consisting of SEQ ID Nos.: 29 and 30.

A linker sequence in the context of the present invention means a sequence between the functional domains. The length of the linker is preferably 1 to 50 or more than 50 amino acids.

Preferably, the present invention relates to a fusion polypeptide according to the present invention, wherein the substrate to which the substrate binding domain binds non-covalently is DNA or RNA having a length of 3 to 3,000,000 base pairs, preferably a length of 3,000 to 30,000 base pairs, especially preferably a length of 30,000 to 300,000 base pairs.

Also preferably, the substrate binding domain of the fusion polypeptide according to the invention binds polypeptides having an amino acid sequence length of 5 to 250 amino acids, preferably 20 to 200 amino acids and especially preferably 50 to 150 amino acids.

Moreover preferably, the substrate binding domain of the fusion polypeptide according to the invention binds to a polypeptide, which comprises a polysaccharide tag having a minimal length of a disaccharide, preferably an amylopectin or dextrin tag.

Preferably, the present invention relates to a fusion polypeptide according to the invention, wherein the fusion polypeptide comprises or consists of an amino acid sequence selected from the group consisting of SEQ ID Nos.: 32 to 99 or an amino acid sequence having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to an amino acid sequence selected from the group consisting of SEQ ID Nos.: 32 to 99.

The SEQ ID NOs.: 32 to 99 are sequences of fusion polypeptides having the functional domains according to the invention. These fusion polypeptides are suitable for transferring a wide variety of different substrates into a cell.

Preferably, the substrates are selected from the group consisting of antibodies such as bermekimab or clazakizumab and polysaccharide tagged bermekimab or clazakizumab, small molecules such as adamantane or fullerene and polysaccharide tagged adamantane or fullerene, antibiotics such as chloramphenicol or kanamycin and polysaccharaide tagged chloramphenicol and kanamycin, biologics such as insulin or human growth factor and polysaccharide tagged insulin or human growth factor, polypeptides such as melatonin or lipocalin and polysaccharide tagged melatonin or lipocalin, transcription factors such as e.g. zinc finger polypeptides, DNA (such as e.g. plasmid DNA, viral double stranded DNA or linear cDNA), RNA (such as e.g. siRNA, mRNA, or double stranded RNA), or a mixture of DNA and RNA.

Preferably, the present invention relates to a fusion polypeptide, wherein the fusion polypeptide comprises or consists of the five functional domains i) to v), i.e. domains iv) and v) are present and not optional in this case,
preferably wherein the pH-sensitive autoprotease domain comprises or consists of at least one amino acid sequence according to SEQ ID No.: 31 or an amino acid sequence having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to an amino acid sequence according to SEQ ID No.: 31.

The pH-sensitive autoprotease is capable of releasing the peptide as a response to the pH-value within the cell. Preferably, the pH-sensitive autoprotease is active at a pH-value of between 6.8 and 7.2.

Another aspect of the present invention relates to a recombinant nucleic acid molecule encoding a fusion polypeptide according to the invention.

Preferably, the cell penetrating peptide domain of the fusion polypeptide according to the invention is encoded by a nucleic acid molecule selected from the group consisting of SEQ ID NOs.: 100 to 123 or a nucleic acid molecule having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to a nucleic acid molecule selected from the group consisting of SEQ ID NOs.: 100 to 123.

Preferably, the substrate binding domain of the fusion polypeptide according to the invention is encoded by a nucleic acid molecule selected from the group consisting of SEQ ID NOs.: 124 and 125 or a nucleic acid molecule having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to a nucleic acid molecule selected from the group consisting of SEQ ID NOs.: 124 and 125.

Preferably, the signal sequence domain of the fusion polypeptide according to the invention is encoded by a nucleic acid molecule selected from the group consisting of SEQ ID NOs.: 126 and 127 or a nucleic acid molecule having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to a nucleic acid molecule selected from the group consisting of SEQ ID NOs.: 126 and 127.

Preferably, the fusion polypeptide according to the invention comprises a linker domain, which is encoded by a nucleic acid molecule selected from the group consisting of SEQ ID NOs.: 128 and 129 or a nucleic acid molecule having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to a nucleic acid molecule selected from the group consisting of SEQ ID NOs.: 128 and 129.

Preferably, the fusion polypeptide according to the invention comprises a pH-sensitive autoprotease domain, which is encoded by a nucleic acid molecule according to SEQ ID NO.: 130 or a nucleic acid molecule having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to a nucleic acid molecule according to SEQ ID NO.: 130.

Preferably, the fusion polypeptide according to the invention is encoded by a nucleic acid molecule selected from the group consisting of SEQ ID NOs.: 131 to 250 or a nucleic acid molecule having at least 90%, preferably at least 91%, preferably at least 92%, preferably at least 93%, preferably at least 94%, preferably at least 95%, preferably at least 96%, preferably at least 97%, preferably at least 98% preferably at least 99% sequence identity to a nucleic acid molecule selected from the group consisting of SEQ ID NOs.: 131 to 250.

Another aspect of the present invention relates to a genetically modified cell, including a recombinant nucleic acid molecule according to the invention, wherein the cell is capable of expressing a fusion polypeptide according to the invention, preferably wherein the cell is selected from the group consisting of Escherichia coli, Vibrio natrigens, Saccheromyces cerevisiae, Aspergillus niger, green algae, microalgae, HEK T293 and Chinese hamster ovary cells (CHO).

Yet another aspect of the present invention relates to a method for producing a fusion polypeptide according to the invention, comprising the steps of
a) providing a genetically modified cell according to the invention;
b) culturing the cell under conditions suitable for expression of a fusion polypeptide according to the invention;
c) obtaining the fusion polypeptide and optionally, unfolding of the obtained fusion polypeptide and directed refolding of said fusion polypeptide.

Step (a) comprises providing a genetically modified cell expressing a fusion polypeptide according to the invention. Such cell is obtainable by introducing a nucleic acid molecule including a sequence encoding a fusion polypeptide according to the invention, preferably in the form of a vector, into the cell by known methods such as for example by transfection or transformation.

In step (b), the cell is cultured under conditions suitable for expressing a fusion polypeptide according to the invention, preferably in a high-density culture. Culture conditions and especially conditions to achieve a high-density culture and corresponding media are well known to the person skilled in the art. Preferably, the expression of the fusion polypeptide is achieved with a subsequent transport to inclusion bodies using a suitable signal sequence according to the invention.

Step (c) comprises obtaining the fusion polypeptide from the culture broth and optionally unfolding of the obtained fusion polypeptide and directed refolding of said fusion polypeptide, if the fusion polypeptide is present in inclusion bodies.

Solubilization conditions for the processing of inclusion bodies and conditions for the directed refolding are well known in the art. Preferably, inclusion bodies are solubilized by using 6 M guanidinium chloride, 8 M urea or 2% sodium dodecyl sulfate and are refolded under neutral or mildly basic conditions.

Preferably, the fusion polypeptide is produced as described in the application PCT/EP2023/053466.

One aspect of the present invention relates to a method for introducing a substrate into a cell, comprising the steps of
a) providing a fusion polypeptide according to the invention, preferably obtained by a method according to the invention;
b) contacting the fusion polypeptide according to the invention with the substrate, which should be introduced into the cell;
c) obtaining a fusion polypeptide with a substrate, which is bond non-covalently to the substrate binding domain of the fusion polypeptide;
d) contacting the obtained fusion polypeptide/substrate complex obtained in step c) with the cell, in which the substrate should be introduced.
e) obtaining a cell with an introduced substrate.

Preferably, the method is performed ex vivo.

Preferably the method comprises the steps of:
(i) Cultivating a genetically modified cell according to the invention expressing a fusion polypeptide according to the invention;
(ii) Purifying the expressed fusion polypeptide obtained in step (i);
(iii) Contacting the expressed fusion polypeptide with the substance, which should be introduced, preferably with DNA, RNA or a mixture thereof;
(iv) Contacting the mixture obtained in step (iii) with the cell in which the substance should be introduced;
(v) Obtaining a cell with an introduced substance, preferably a transformed cell, wherein the steps (i) to (iv) are performed ex vivo, preferably in vitro.

Preferably, the step of contacting b) or iv) is performed at a temperature of 20 to 37° C. and a pH value of between 6 and 9 in the presence of a buffer selected from TRIS, HEPES, PIPES or a buffer containing sodium acetate or arginine and sucrose.

Also preferably, the contacting step d) or iv) is done using the low pressure spray method (LPS), if a plant cell is to be transformed or a substance is to be introduced into a plant cell. Preferably, if a substance should be introduced in other organisms than plants, the mixture obtained in step (iii) is contacted with the cell in the presence of a buffer, which is ideal for the targeted cell.

The person skilled in the art is aware of suitable buffers for specific cells and organisms.

Another aspect relates to a kit for transforming cells, comprising at least one fusion polypeptide according the invention.

Preferably, the kit is a transformation kit and further comprises buffers and reagents suitable for the transformation of the targeted cells. Suitable buffers and reagents are known by the person skilled in the art.

Another aspect of the present invention related to a non-therapeutic use of a fusion polypeptide according to the invention for introducing a substance into a target cell, wherein the substance is selected from the group consisting of antibodies, small molecules, antibiotics, biologics, polypeptides, transcription factors or DNA, RNA or a mixture of DNA and RNA.

Preferably, the substrate is selected from the group consisting of antibodies such as bermekimab or clazakizumab and polysaccharide tagged bermekimab or clazakizumab, small molecules such as adamantane or fullerene and polysaccharide tagged adamantane or fullerene, antibiotics such as chloramphenicol or kanamycin and polysaccharaide tagged chloramphenicol and kanamycin, biologics such as insulin or human growth factor and polysaccharide tagged insulin or human growth factor, polypeptides such as melatonin or lipocalin and polysaccharide tagged melatonin or lipocalin, transcription factors such as e.g.

zinc finger polypeptides, DNA (such as e.g. plasmid DNA, viral double stranded DNA or linear cDNA), RNA (such as e.g. siRNA, mRNA, or double stranded RNA), or a mixture of DNA and RNA.

Another aspect of the invention relates to the non-therapeutic use of a fusion polypeptide according to the invention, wherein the target cell is selected from the group consisting of plant cells, bacterial cells, mammalian cells and yeast cells, preferably plant cells.

Further aspects and advantages of the invention result from the subsequent description of preferred examples.

### SHORT DESCRIPTION OF SEQUENCES

SEQ ID NOs.: 1 to SEQ ID NO. 24 describe amino acid sequences encoding the artificial cell penetrating peptide 1 to 24.

SEQ ID NOs.: 25 and 26 describe amino acid sequences encoding binding domains.

SEQ ID NOs.: 27 and 28 describe amino acid sequences encoding signal sequences.

SEQ ID NO.: 29 and 30 describe amino acid sequences encoding linker domains.

SEQ ID NO.: 31 describes an amino acid sequence encoding a pH-sensitive autoprotease.

SEQ ID NOs.: 32 to 99 describe amino acid sequences encoding fusion polypeptides according to the invention.

SEQ ID NOs.: 100 to 123 describe nucleic acid sequences encoding the artificial cell penetrating peptide 1 to 24.

SEQ ID NOs.: 124 and 125 describe nucleic acid sequences encoding binding domains.

SEQ ID NOs.: 126 and 127 describe nucleic acid sequences encoding linker domains.

SEQ ID NOs.: 128 and 129 describe nucleic acid sequences encoding linker domains.

SEQ ID NO.: 130 describes a nucleic acid domain encoding a pH-sensitive autoprotease.

SEQ ID No.: 131 to 250 describe nucleic acid sequences encoding fusion polypeptides according to the invention.

### SHORT DESCRIPTION OF FIGURES

FIG. 1: Graph showing the mean value of the GFP silencing ratio of all silenced leaves originating from the treated strain apical meristem with regard to the total leaf area. The results of LPS treatment with transportan-, R₉-transportan-, R₅A₄-transportan-, S4₁₃PV-, R₉-S4₁₃PV-and KLAVH₁₆-22nt siRNA complex in the molar RNA: CPP ratios 5:1, 10:1, 50:1, as well as the overall result (combined) are shown. The 10% CPD-22nt siRNA complex control is shown under the category ‘combined’. The number of leaves evaluated depends on the individual and ranges from 1 to 6.

FIG. 2: Number of GFP silenced flowers developed by CPP-22nt siRNA complex LPS treated N. benthamiana 16C of the first offspring generation (F1). The applied siRNA concentration was 14 ng μl-1.

FIG. 3: Relative amount of silenced offspring in the F1 generation with regard to the parental plants. Each line (sample) is derived from a single treated mother plant. Numbers at the end of the cell line name indicate the applied molar siRNA/CPP ratio (e.g. 50=50:1). Seeds were germinated on 0.5×MS medium under sterile conditions, n=200.

FIG. 4: Panel A shows the colony growth on an agar plate of E. coli BL21, which were incubated without applying a heat shock together with fusion polypeptide according to the invention having the binding domain according to SEQ ID NO.: 25 and a cell penetrating peptide according to SEQ ID NO.: 18. Panel B shows the colony growth on an agar plate of E. coli BL21, which were incubated with applying a heat shock together with fusion polypeptide according to the invention having the binding domain according to SEQ ID NO.: 25 and a cell penetrating peptide according to SEQ ID NO.: 18. Panel C shows the colony growth on an agar plate of E. coli BL21, after incubating the cells with the plasmid without applying a heat shock. Panel C shows the colony growth on an agar plate of E. coli BL21, after incubating the cells with the plasmid with applying a heat shock. The agar plates comprise the antibiotics kanamycin and chloramphenicol, wherein the plasmids carry a gene conveying a resistance to kanamycin and chloramphenicol.

FIG. 5: Gel showing the digestion of the plasmids that were present in the E. coli cells, which were grown on antibiotic supplemented agar plates according to FIG. 4. F1 depicts the digestion of the plasmid as found in colonies according to panel A, F2 depicts the digestion of the plasmid as found in colonies according to panel B, F3 depicts the digestion of the plasmid as found in colonies according to panel C, F4 depicts the digestion of the plasmid as found in colonies according to panel

D. The right band depicts the 1 kb DNA marker.

### EXAMPLES

### Example 1

### Treatment of Plants with siRNA Fusion Polypeptide Complexes

The fusion polypeptides according to SEQ ID NO.: 67, 68, 69, 52, 53, 48 and 72 were subjected to a DNase I digestion for eliminating remaining DNA from the subsequent purification steps at 37° C. for 15 minutes. The DNase I was deactivated for 10 minutes at 65° C. Thereafter, the siRNA was brought in contact with the fusion polypeptide sample. The siRNA concentration was 14 ng/ul and the molar ratios of siRNA to CPP carrying fusion polypeptide were 5:1, 10:1 and 50:1. They were mixed at 1100 rpm for 2 hours at room temperature in the presence of 0.1% Silwet® L-77.

In one aspect, low pressure spraying (LPS) was used for plant treatment. This method involves spraying the siRNA: fusion polypeptide complex at 1.3 bar onto N. benthamiana in nanomolar amounts (ratio 1:10 siRNA: fusion polypeptide) per 100 to 500 μL buffer. The N. benthamiana plants express GFP transgenously, which is silenced by the introduced siRNA.

The plants were prepared by incubating them in a highly humid environment at a humidity of 60 to 100% for one hour. The spraying of the leaves and/or the stems of the plant was performed using a Conrad™ HP-200 airbrush pistol. The application of the siRNA: fusion polypeptide mixture was performed with juvenile plants at an age of two to fourteen days after the end of the embryonal phase as subjects of the treatment. The juvenile plants were treated. The organs that were treated were the meristem, the leaves themselves and the leaf edges.

The siRNAs that were used to produce the results were RNA sequences having a length of 22 amino acids. The fusion polypeptides that were used were designed with the following cell penetrating peptides: transportan-(SEQ ID No.: 16), R₉-transportan (SEQ ID No.: 17), R₅A₄-transportan (SEQ ID NO.: 18), S₄₁₃PV (SEQ ID NO.: 1), R₉-S4₁₃PV (SEQ ID NO.: 2), and KLAVH₁₆ (SEQ ID NO.: 21). Accordingly, fusion polypeptides with SEQ ID NO.: 67, 68, 69, 52, 53, 48 and 72 were used in the experiments.

The results were measured as GFP silencing ratio of all silenced leaves originating from the apical medical stem. Results were compared with the method of introducing siRNA to cells using carbon polymer dots (as described in Schwartz et al., 2020, “Carbon Dots for Efficient Small Interfering RNA Delivery and Gene Silencing in Plants”).

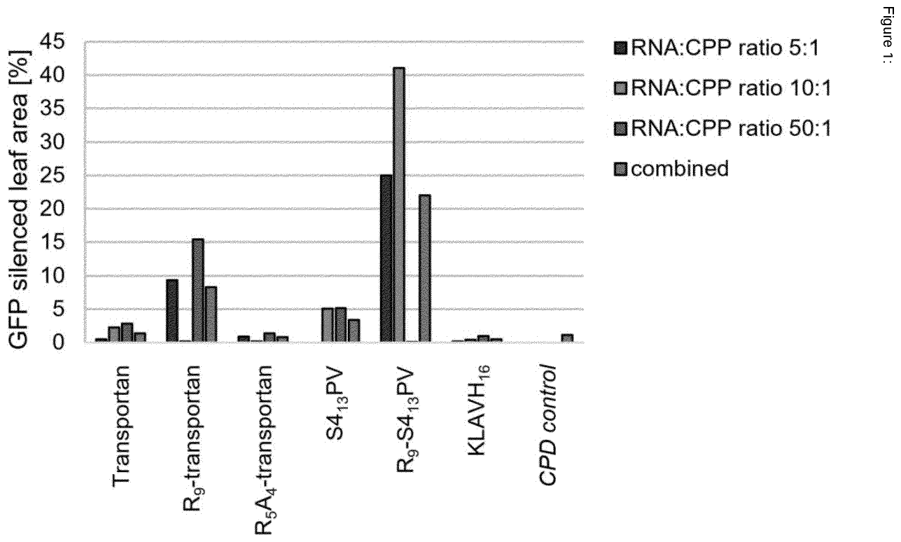

In FIG. 1, the results of the gene silencing experiments are displayed for the parental generation. The results of LPS treatment with transportan-, R₉-transportan-, R₅A₄-transportan-, S4₁₃PV-, R₉-S4₁₃PV- and KLAVH₁₆-22nt siRNA complex in the molar RNA: CPP-fusion polypeptide ratios 5:1, 10:1, 50:1, as well as the overall result (combined) are shown. The results of the 10% CPD (carbon polymer dots)-22nt siRNA complex control is shown under the bar ‘combined’ as only one fixed ratio was applied. The number of leaves evaluated depends on the individual and ranges from 1 to 6.

The first offspring generation (F1) of treated plants was also investigated for GFP silencing. It could be found that silencing was observed in cases where fusion polypeptides according to the invention designed with cell penetrating peptides were used to carry 22nt siRNA inside the target cells, silencing also occurred in the F1 generation (first offspring generation). The applied concentration of siRNA was 14 ng μl⁻¹.

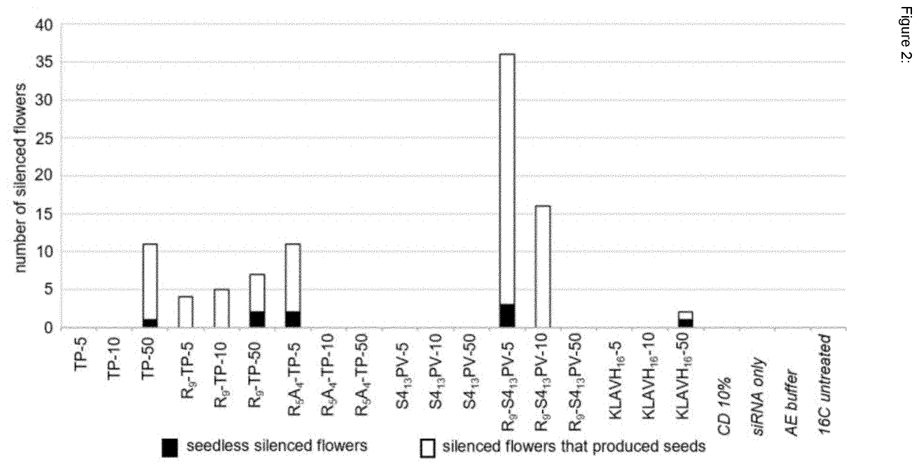

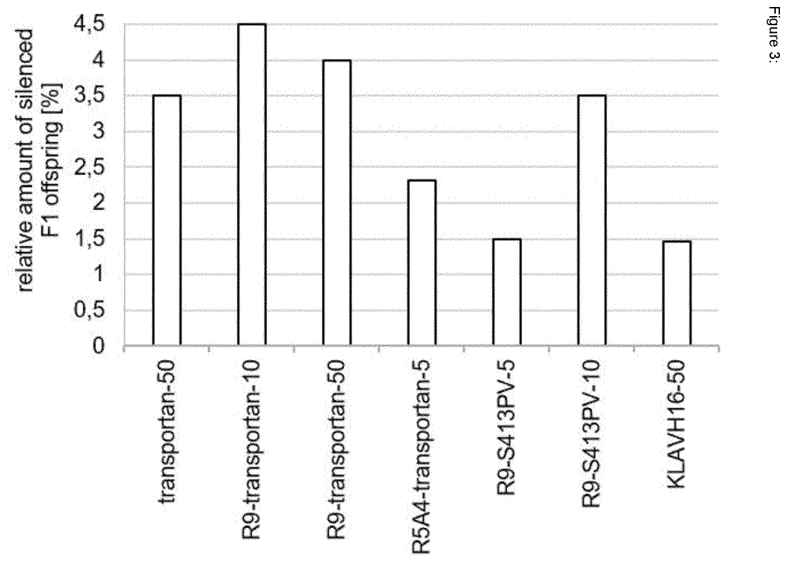

The results are depicted in FIG. 2 and shows that the siRNA delivery with different fusion polypeptides according to the invention designed with cell penetrating peptides, several plants (N. benthamiana) exhibit silenced GFP levels also in the first offspring generation F1, while maintaining the seed production capacity. The relative amount of silenced plants in the F1 generation to the parental generation is shown in FIG. 3. The numbers at the end of the sample names indicate the applied molar siRNA/CPP ratio (e.g. 50=50:1). Seeds were germinated on 0.5×MS medium under sterile conditions with a sample size of 200.

### Example 2

### Transfection of Tobacco Leaves for Gene Silencing

Any one of the fusion polypeptides according to SEQ ID NO.: 32 to 99 can be used as a transport vehicle for introduction DNA into plant leaves. In the following, a fusion polypeptide according to SEQ ID NO.: 67, 68, 69, 52, 53, 48 and 72 is used.

Step 1) A sample of the fusion polypeptide is adjusted to a pH of between pH 7.7 to pH 8.3.

Step 2) An aliquot of 80 μl of DNase I with 1000U is added to a sample of more than one gram fusion polypeptide for eliminating remaining DNA from the subsequent purification steps. The mixture is mildly agitated at 37° C. for 30 minutes. Then, the sample is relaxed at room temperature for at least 30 minutes. Then, the DNase I is incubated at 65° C. for 30 minutes.

Step 3) The concentration of the fusion polypeptide is determined photometrically and the fusion polypeptide is mixed at a desired ratio with the desired siRNA in a buffer with pH above 7.2. The sample is incubates at room temperature without agitation for at least ten minutes.

Step 4) The mixture is sprayed on growing plants that were grown on a suitable medium and are observed for several days.

The treatment led to systemic gene silencing which means that the effect was not limited to the infiltration spot.

### Example 3

### Transformation of E. coli Using a Fusion Polypeptide According to the Invention (SEQ ID NO.: 67, 68, 69, 52, 53, 48 and 72)

The method using the fusion polypeptide according to the invention and a known heat shock protocol for transformation of E. coli cells were compared:

Step 1) Four 100 μl Calcium competent BL21 E. coli cells were thawed at 4° C. in a refrigerator for 15 minutes. Four LB agarplates with 15 g/L agarose, 30 mg/L kanamycin and 30 mg/L chloramphenicol were incubated at 37° C. together with 4 mL SOC (Super optimal broth with catabolite repression) medium.

Step 2) After thawing, the cells are contacted with either 500 ng of pure plasmid DNA or 500 ng of plasmid DNA with a fusion polypeptide according to SEQ ID NO.: SEQ ID NO.: 67, 68, 69, 52, 53, 48 and 72 carrying additionally an autoprotease domain according to SEQ ID NO.: 31, that was mixed with the plasmid DNA at a rate of 1:10 in favour of the DNA as carrier of known protein concentration. The plasmid comprises a resistance against chloramphenicol and kanamycin.

Step 3) The cells of step 2) are incubated at 4° C.

Step 4) After the incubation at 4° C. one of each of the two vials of cells with the different DNA applications according to step 2 is subjected to a heat shock for one minute at 42° C. and then relaxed at 4° C. for 2 minutes. The other two vials are left at 4° C. for the same amount of time.

Step 5) After heat shock treatment or no treatment respectively, all four vials are treated with 900 μl of warm SOC medium and agitated for one hour at 37° C.

Step 6) One aliquot of the cells is transferred to the warm LB agar plates which are incubated over night at 37° C.

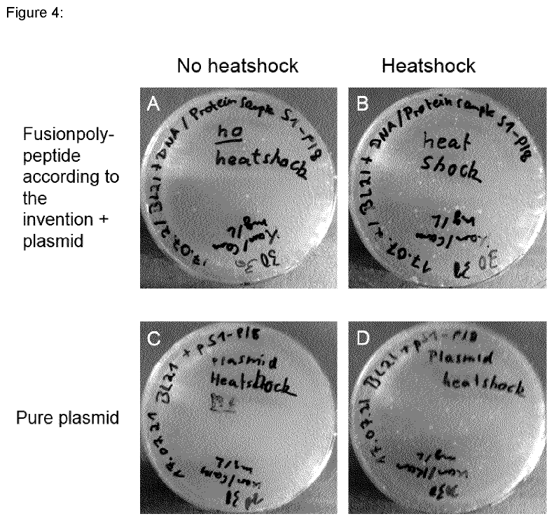

Step 7) The growth on all plates was evaluated and can be seen as summarized in Table 1 and in FIG. 4.

TABLE 1
Summary of comparison of transformation protocols

Heatshock

| DNA delivering | Heatshock | No heatshock |
|---|---|---|
| Without fusion polypeptide | Growth | No growth |
| according to the invention | | |
| With fusion polypeptide | Growth | Growth |
| according to the invention | | |

The results show that the fusion polypeptide system was able to a) pick up plasmid DNA and b) actively transport is into the cell when penetrating the membranes without making use of the transport system of the cell.

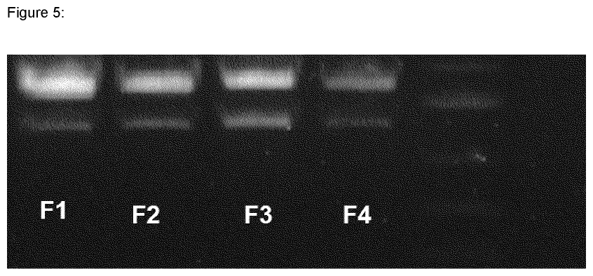

It was determined if the cells that grow on the antibiotic-supplemented agar plates carry the antibiotic resistance by extracting the DNA of the grown colonies and digesting the plasmid. It could be proven that the resistance gene is present in all grown colonies (FIG. 5), wherein the highest amount could be extracted from the cells, which were transformed by using the fusion polypeptides according to the invention.

1. A fusion polypeptide comprising at least three functional domains:
i) a substrate binding domain binding to a substrate non-covalently, wherein the substrate is selected from the group consisting of polypeptides, DNA, RNA or a mixture of DNA and RNA and
wherein the substrate binding domain comprises at least one amino acid sequence selected from the group consisting of SEQ ID NOs.: 25 and 26 or an amino acid sequence having at least 90% sequence identity to an amino acid sequence selected from the group consisting of SEQ ID NOs.: 25 and 26;
ii) a cell penetrating domain;
iii) a signal sequence;
iv) optionally a linker domain; and
v) optionally a pH-sensitive autoprotease domain,
wherein the cell penetrating domain has a length of between 15 and 50 amino acid residues, and
wherein the cell penetrating domain comprises at least 70% amino acid residues with a helix potential value >1, dependent on the overall length of the cell penetrating domain.

2. The fusion polypeptide according to claim 1, wherein the cell penetrating domain comprises or consists of at least one amino acid sequence selected from the group consisting of SEQ ID NOs.: 1 to 24, or an amino acid sequence having a sequence identity of more than 90% to an amino acid sequence selected from the group consisting of SEQ ID NOs.: 1 to 24.

3. The fusion polypeptide according to claim 1, wherein the signal sequence is an inclusion body promoting sequence or a secretion sequence.

4. The fusion polypeptide according to claim 1, wherein the signal sequence comprises at least one amino acid sequence selected from the group consisting of SEQ ID NOs.: 27 and 28 or an amino acid sequence having at least 90% sequence identity to an amino acid sequence selected from the group consisting of SEQ ID NOs.: 27 and 28.

5. The fusion polypeptide according to claim 1, wherein the fusion polypeptide comprises a linker domain and, wherein
the linker domain comprises an N-terminal alpha helix and/or a C-terminal sequence of a random coil structure, wherein the linker domain comprises at least one amino acid sequence selected from the group consisting of SEQ ID NOs.: 29 and 30 or an amino acid sequence having at least 90% sequence identity to an amino acid sequence selected from the group consisting of SEQ ID NOs.: 29 and 30.

6. The fusion polypeptide according to claim 1, wherein the substrate to which the substrate binding domain binds non-covalently is DNA or RNA having a length of 3 to 3,000,000 base pairs.

7. The fusion polypeptide according to claim 1, wherein the fusion polypeptide comprises an amino acid sequence selected from the group consisting of SEQ ID NOs.: 32 to 99 or an amino acid sequence having at least 90% sequence identity to an amino acid sequence selected from the group consisting of SEQ ID NOs.: 32 to 99.

8. The fusion polypeptide according to claim 1, wherein the fusion polypeptide comprises er consists of the five functional domains i) to v), preferably wherein the pH-sensitive autoprotease domain comprises or consists of at least one amino acid sequence according to SEQ ID No.: 31 or an amino acid sequence having at least 90% sequence identity to an amino acid sequence according to SEQ ID No.: 31.

9. A recombinant nucleic acid molecule encoding a fusion polypeptide according to claim 1.

10. A genetically modified cell, comprising a recombinant nucleic acid molecule according to claim 9.

11. A method for introducing a substance into a cell, comprising the steps of:
(i) Cultivating a genetically modified cell according to claim 10 expressing the fusion polypeptide according to claim 1;
(ii) Purifying the expressed fusion polypeptide obtained in step (i);
(iii) Contacting the expressed fusion polypeptide of step (ii) with the substance, which should be introduced;
(iv) Contacting the mixture obtained in step (iii) with the cell in which the substance should be introduced;
(v) Obtaining a cell with an introduced substance,
wherein the steps (i) to (iv) are performed ex vivo.

12. The method according to claim 11, wherein the cell, in which the substance is introduced, is selected from eukaryotic and prokaryotic organisms and wherein the contacting in step (iv) is done at a temperature of between 2° C. and 40° C. for a time of between 10 min to 360 min.

13. A kit for transforming cells, comprising at least one fusion polypeptide according to claim 1.

14. The method of claim 11, wherein the substance is selected from the group consisting of antibodies, small molecules, antibiotics, biologics, polypeptides, transcription factors or DNA, RNA or a mixture of DNA and RNA.

15. The method of claim 11, wherein the cell is selected from the group consisting of plant cells, bacterial cells, mammalian cells and yeast cels.


SEQUENCE LISTING (252 sequences)

SEQ ID NO: 1 - protein, 21 aa, synthetic construct
CVKRKKKPAKLVKKLLTKWLA

SEQ ID NO: 2 - protein, 30 aa, synthetic construct
CVKRKKKPAKLVKKLLTKWLARRRRRRRRR

SEQ ID NO: 3 - protein, 30 aa, synthetic construct
CVKRKKKPAKLVKKLLTKWLARARARARAR

SEQ ID NO: 4 - protein, 18 aa, synthetic construct
QQRKRKIWSILAPLGTTL

SEQ ID NO: 5 - protein, 27 aa, synthetic construct
QQRKRKIWSILAPLGTTLRRRRRRRRR

SEQ ID NO: 6 - protein, 27 aa, synthetic construct
QQRKRKIWSILAPLGTTLRARARARAR

SEQ ID NO: 7 - protein, 18 aa, synthetic construct
LTTGLPALISWIKRKRQQ

SEQ ID NO: 8 - protein, 27 aa, synthetic construct
LTTGLPALISWIKRKRQQRRRRRRRRR

SEQ ID NO: 9 - protein, 27 aa, synthetic construct
LTTGLPALISWIKRKRQQRARARARAR

SEQ ID NO: 10 - protein, 25 aa, synthetic construct
QQRKRKWSILAPLGTTLPKKKRKVC

SEQ ID NO: 11 - protein, 34 aa, synthetic construct
QQRKRKWSILAPLGTTLPKKKRKVCRRRRRRRRR

SEQ ID NO: 12 - protein, 34 aa, synthetic construct
QQRKRKWSILAPLGTTLPKKKRKVCRARARARAR

SEQ ID NO: 13 - protein, 25 aa, synthetic construct
CVKRKKKPLTTGLPALISWKRKRQQ

SEQ ID NO: 14 - protein, 34 aa, synthetic construct
CVKRKKKPLTTGLPALISWKRKRQQRRRRRRRRR

SEQ ID NO: 15 - protein, 34 aa, synthetic construct
CVKRKKKPLTTGLPALISWKRKRQQRARARARAR

SEQ ID NO: 16 - protein, 27 aa, synthetic construct
LIKKALAALAKLNIKGLLYGASNLTWG

SEQ ID NO: 17 - protein, 36 aa, synthetic construct
LIKKALAALAKLNIKGLLYGASNLTWGRRRRRRRRR

SEQ ID NO: 18 - protein, 36 aa, synthetic construct
LIKKALAALAKLNIKGLLYGASNLTWGRARARARAR

SEQ ID NO: 19 - protein, 21 aa, synthetic construct
HVALKHVALKHVALKHVALKW

SEQ ID NO: 20 - protein, 30 aa, synthetic construct
HVALKHVALKHVALKHVALKWRRRRRRRRR

SEQ ID NO: 21 - protein, 30 aa, synthetic construct
HVALKHVALKHVALKHVALKWRARARARAR

SEQ ID NO: 22 - protein, 26 aa, synthetic construct
RVLKQARVLKQARVLKQARVLKKQHW

SEQ ID NO: 23 - protein, 35 aa, synthetic construct
RVLKQARVLKQARVLKQARVLKKQHWRRRRRRRRR

SEQ ID NO: 24 - protein, 35 aa, synthetic construct
RVLKQARVLKQARVLKQARVLKKQHWRARARARAR

SEQ ID NO: 25 - protein, 540 aa, synthetic construct
VYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 26 - protein, 268 aa, synthetic construct
VYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNN

SEQ ID NO: 27 - protein, 25 aa, synthetic construct
MAVLKTRRSTRRSTRRAVLKDWEAM

SEQ ID NO: 28 - protein, 32 aa, synthetic construct
MVEDPHRLKRLKRLVANARARARARLVNDIEN

SEQ ID NO: 29 - protein, 42 aa, synthetic construct
GSKESGSVSSEQLAQFRSLDAGSASELNHFELLYKTNKQKPV

SEQ ID NO: 30 - protein, 14 aa, synthetic construct
ALVTALVTALVTTS

SEQ ID NO: 31 - protein, 142 aa, synthetic construct
GVEEYCYNISGRPVIGDPTEVYPDNISKVPHDKGRGNIGTSIRNLPRRGNCRVCYSGQYIGIYGPVFYQDVANAELPRMLVEHKTKICHVVKRIGRVTGSLYHVLKCINGCNVIKYSRCGLEPSSLKWIRNFTDCPLWVTSC

SEQ ID NO: 32 - protein, 334 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSQQRKRKIWSILAPLGTTLRRRRRRRRR

SEQ ID NO: 33 - protein, 334 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSQQRKRKIWSILAPLGTTLRARARARAR

SEQ ID NO: 34 - protein, 325 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSLTTGLPALISWIKRKRQQ

SEQ ID NO: 35 - protein, 334 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSLTTGLPALISWIKRKRQQRRRRRRRRR

SEQ ID NO: 36 - protein, 334 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSLTTGLPALISWIKRKRQQRARARARAR

SEQ ID NO: 37 - protein, 332 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSQQRKRKWSILAPLGTTLPKKKRKVC

SEQ ID NO: 38 - protein, 341 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSQQRKRKWSILAPLGTTLPKKKRKVCRRRRRRRRR

SEQ ID NO: 39 - protein, 341 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSQQRKRKWSILAPLGTTLPKKKRKVCRARARARAR

SEQ ID NO: 40 - protein, 332 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSCVKRKKKPLTTGLPALISWKRKRQQ

SEQ ID NO: 41 - protein, 341 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSCVKRKKKPLTTGLPALISWKRKRQQRRRRRRRRR

SEQ ID NO: 42 - protein, 341 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSCVKRKKKPLTTGLPALISWKRKRQQRARARARAR

SEQ ID NO: 43 - protein, 332 aa, synthetic construct; identical to SEQ ID NO: 40

SEQ ID NO: 44 - protein, 341 aa, synthetic construct; identical to SEQ ID NO: 41

SEQ ID NO: 45 - protein, 341 aa, synthetic construct; identical to SEQ ID NO: 42

SEQ ID NO: 46 - protein, 328 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSHVALKHVALKHVALKHVALKW

SEQ ID NO: 47 - protein, 337 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSHVALKHVALKHVALKHVALKWRRRRRRRRR

SEQ ID NO: 48 - protein, 337 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSHVALKHVALKHVALKHVALKWRARARARAR

SEQ ID NO: 49 - protein, 333 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSRVLKQARVLKQARVLKQARVLKKQHW

SEQ ID NO: 50 - protein, 342 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSRVLKQARVLKQARVLKQARVLKKQHWRRRRRRRRR

SEQ ID NO: 51 - protein, 342 aa, synthetic construct
MAVLKTRRSTRRSTRRAVLKDWEAMVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNVYVKPVYVKVYVPNIHYWNTTGTTTGTTWPTAMKEEGEEIYSIIIPSSSGDVSIIIPNIHYWNTTGTTTWPTAMKEIYSIIIPSSSGDVSIIIPNIHYWNTTGTTWPAMKEDGEIYSIIIPSGDVSIIINDGNNALVTALVTALVTTSRVLKQARVLKQARVLKQARVLKKQHWRARARARAR

SEQ ID NO: 52 - protein, 600 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSCVKRKKKPAKLVKKLLTKWLA

SEQ ID NO: 53 - protein, 609 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSCVKRKKKPAKLVKKLLTKWLARRRRRRRRR

SEQ ID NO: 54 - protein, 609 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSCVKRKKKPAKLVKKLLTKWLARARARARAR

SEQ ID NO: 55 - protein, 597 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSQQRKRKIWSILAPLGTTL

SEQ ID NO: 56 - protein, 606 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSQQRKRKIWSILAPLGTTLRRRRRRRRR

SEQ ID NO: 57 - protein, 606 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSQQRKRKIWSILAPLGTTLRARARARAR

SEQ ID NO: 58 - protein, 597 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSLTTGLPALISWIKRKRQQ

SEQ ID NO: 59 - protein, 606 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSLTTGLPALISWIKRKRQQRRRRRRRRR

SEQ ID NO: 60 - protein, 606 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSLTTGLPALISWIKRKRQQRARARARAR

SEQ ID NO: 61 - protein, 604 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSQQRKRKWSILAPLGTTLPKKKRKVC

SEQ ID NO: 62 - protein, 613 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSQQRKRKWSILAPLGTTLPKKKRKVCRRRRRRRRR

SEQ ID NO: 63 - protein, 613 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSQQRKRKWSILAPLGTTLPKKKRKVCRARARARAR

SEQ ID NO: 64 - protein, 604 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSCVKRKKKPLTTGLPALISWKRKRQQ

SEQ ID NO: 65 - protein, 613 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSCVKRKKKPLTTGLPALISWKRKRQQRRRRRRRRR

SEQ ID NO: 66 - protein, 613 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSCVKRKKKPLTTGLPALISWKRKRQQRARARARAR

SEQ ID NO: 67 - protein, 606 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSLIKKALAALAKLNIKGLLYGASNLTWG

SEQ ID NO: 68 - protein, 615 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSLIKKALAALAKLNIKGLLYGASNLTWGRRRRRRRRR

SEQ ID NO: 69 - protein, 615 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSLIKKALAALAKLNIKGLLYGASNLTWGRARARARAR

SEQ ID NO: 70 - protein, 600 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSHVALKHVALKHVALKHVALKW

SEQ ID NO: 71 - protein, 609 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSHVALKHVALKHVALKHVALKWRRRRRRRRR

SEQ ID NO: 72 - protein, 609 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSHVALKHVALKHVALKHVALKWRARARARAR

SEQ ID NO: 73 - protein, 605 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSRVLKQARVLKQARVLKQARVLKKQHW

SEQ ID NO: 74 - protein, 614 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSRVLKQARVLKQARVLKQARVLKKQHWRRRRRRRRR

SEQ ID NO: 75 - protein, 614 aa, synthetic construct
MAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNALVTALVTALVTTSRVLKQARVLKQARVLKQARVLKKQHWRARARARAR

SEQ ID NO: 76 - protein, 586 aa, synthetic construct
CVKRKKKPAKLVKKLLTKWLAMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 77 - protein, 595 aa, synthetic construct
CVKRKKKPAKLVKKLLTKWLARRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 78 - protein, 595 aa, synthetic construct
CVKRKKKPAKLVKKLLTKWLARARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 79 - protein, 583 aa, synthetic construct
QQRKRKIWSILAPLGTTLMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 80 - protein, 592 aa, synthetic construct
QQRKRKIWSILAPLGTTLRRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 81 - protein, 592 aa, synthetic construct
QQRKRKIWSILAPLGTTLRARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 82 - protein, 583 aa, synthetic construct
LTTGLPALISWIKRKRQQMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 83 - protein, 592 aa, synthetic construct
LTTGLPALISWIKRKRQQRRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 84 - protein, 592 aa, synthetic construct
LTTGLPALISWIKRKRQQRARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 85 - protein, 590 aa, synthetic construct
QQRKRKWSILAPLGTTLPKKKRKVCMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 86 - protein, 599 aa, synthetic construct
QQRKRKWSILAPLGTTLPKKKRKVCRRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 87 - protein, 599 aa, synthetic construct
QQRKRKWSILAPLGTTLPKKKRKVCRARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 88 - protein, 590 aa, synthetic construct
CVKRKKKPLTTGLPALISWKRKRQQMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 89 - protein, 599 aa, synthetic construct
CVKRKKKPLTTGLPALISWKRKRQQRRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 90 - protein, 599 aa, synthetic construct
CVKRKKKPLTTGLPALISWKRKRQQRARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 91 - protein, 592 aa, synthetic construct
LIKKALAALAKLNIKGLLYGASNLTWGMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 92 - protein, 601 aa, synthetic construct
LIKKALAALAKLNIKGLLYGASNLTWGRRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 93 - protein, 601 aa, synthetic construct
LIKKALAALAKLNIKGLLYGASNLTWGRARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 94 - protein, 586 aa, synthetic construct
HVALKHVALKHVALKHVALKWMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 95 - protein, 595 aa, synthetic construct
HVALKHVALKHVALKHVALKWRRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 96 - protein, 595 aa, synthetic construct
HVALKHVALKHVALKHVALKWRARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 97 - protein, 591 aa, synthetic construct
RVLKQARVLKQARVLKQARVLKKQHWMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 98 - protein, 600 aa, synthetic construct
RVLKQARVLKQARVLKQARVLKKQHWRRRRRRRRRMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 99 - protein, 600 aa, synthetic construct
RVLKQARVLKQARVLKQARVLKKQHWRARARARARMAVLKTRRSTRRSTRRAVLKDWEAMVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNNVYVKVPDTWTGIPNIHYWNTTGGTTTWPGKQMKEEGNEIYSIIIPKSFGDVSIIINDGNN

SEQ ID NO: 100 - DNA, 63 bp, synthetic construct
  source 1..63: mol_type = other DNA
tgcgtgaaacgcaaaaaaaaaccggcgaaactggtgaaaaaactgctgaccaaatggctggcg

SEQ ID NO: 101 - DNA, 90 bp, synthetic construct
  source 1..90: mol_type = other DNA
tgcgtgaaacgcaaaaaaaaaccggcgaaactggtgaaaaaactgctgaccaaatggctggcgcgccgccgccgccgccgccgccgccgc

SEQ ID NO: 102 - DNA, 90 bp, synthetic construct
  source 1..90: mol_type = other DNA
tgcgtgaaacgcaaaaaaaaaccggcgaaactggtgaaaaaactgctgaccaaatggctggcgcgcgcgcgcgcgcgcgcgcgcgcgcgc

SEQ ID NO: 103 - DNA, 51 bp, synthetic construct
  source 1..51: mol_type = other DNA
cagcagcgcaaacgcaaatggagcattctggcgccgctgggcaccaccctg

SEQ ID NO: 104 - DNA, 78 bp, synthetic construct
  source 1..78: mol_type = other DNA
cagcagcgcaaacgcaaatggagcattctggcgccgctgggcaccaccctgcgccgccgccgccgccgccgccgccgc

SEQ ID NO: 105 - DNA, 78 bp, synthetic construct
  source 1..78: mol_type = other DNA
cagcagcgcaaacgcaaatggagcattctggcgccgctgggcaccaccctgcgcgcgcgcgcgcgcgcgcgcgcgcgc

SEQ ID NO: 106 - DNA, 51 bp, synthetic construct
  source 1..51: mol_type = other DNA
ctgaccaccggcctgccggcgctgattagctggaaacgcaaacgccagcag

SEQ ID NO: 107 - DNA, 78 bp, synthetic construct
  source 1..78: mol_type = other DNA
ctgaccaccggcctgccggcgctgattagctggaaacgcaaacgccagcagcgccgccgccgccgccgccgccgccgc

SEQ ID NO: 108 - DNA, 78 bp, synthetic construct
  source 1..78: mol_type = other DNA
ctgaccaccggcctgccggcgctgattagctggaaacgcaaacgccagcagcgcgcgcgcgcgcgcgcgcgcgcgcgc

SEQ ID NO: 109 - DNA, 75 bp, synthetic construct
  source 1..75: mol_type = other DNA
tgcgtgaaacgcaaaaaaaaaccgctgaccaccggcctgccggcgctgattagctggaaacgcaaacgccagcag

SEQ ID NO: 110 - DNA, 102 bp, synthetic construct
  source 1..102: mol_type = other DNA
tgcgtgaaacgcaaaaaaaaaccgctgaccaccggcctgccggcgctgattagctggaaacgcaaacgccagcagcgccgccgccgccgccgccgccgccgc

SEQ ID NO: 111 - DNA, 102 bp, synthetic construct
  source 1..102: mol_type = other DNA
tgcgtgaaacgcaaaaaaaaaccgctgaccaccggcctgccggcgctgattagctggaaacgcaaacgccagcagcgcgcgcgcgcgcgcgcgcgcgcgcgc

SEQ ID NO: 112 - DNA, 76 bp, synthetic construct
  source 1..76: mol_type = other DNA
tgcgtgaaacgcaaaaaaaaaccgctgaccaccggcctgccggcgctgattagctggaaacgcaaacgccagcagc

SEQ ID NO: 113 - DNA, 103 bp, synthetic construct
  source 1..103: mol_type = other DNA
tgcgtgaaacgcaaaaaaaaaccgctgaccaccggcctgccggcgctgattagctggaaacgcaaacgccagcagccgccgccgccgccgccgccgccgccgc

SEQ ID NO: 114 - DNA, 103 bp, synthetic construct
  source 1..103: mol_type = other DNA
tgcgtgaaacgcaaaaaaaaaccgctgaccaccggcctgccggcgctgattagctggaaacgcaaacgccagcagccgcgcgcgcgcgcgcgcgcgcgcgcgc

SEQ ID NO: 115 - DNA, 81 bp, synthetic construct
  source 1..81: mol_type = other DNA
ctgattaaaaaagcgctggcggcgctggcgaaactgaacattaaaggcctgctgtatggcgcgagcaacctgacctggggc

SEQ ID NO: 116 - DNA, 109 bp, synthetic construct
  source 1..109: mol_type = other DNA
ctgattaaaaaagcgctggcggcgctggcgaaactgaacattaaaggcctgctgtatggcgcgagcaacctgacctggggcccgccgccgccgccgccgccgccgccgc

SEQ ID NO: 117 - DNA, 109 bp, synthetic construct
  source 1..109: mol_type = other DNA
ctgattaaaaaagcgctggcggcgctggcgaaactgaacattaaaggcctgctgtatggcgcgagcaacctgacctggggcccgcgcgcgcgcgcgcgcgcgcgcgcgc

SEQ ID NO: 118 - DNA, 63 bp, synthetic construct
  source 1..63: mol_type = other DNA
catgtggatctgaaacatgtggatctgaaacatgtggatctgaaacatgtggatctgaaatgg

SEQ ID NO: 119 - DNA, 91 bp, synthetic construct
  source 1..91: mol_type = other DNA
catgtggatctgaaacatgtggatctgaaacatgtggatctgaaacatgtggatctgaaatggccgccgccgccgccgccgccgccgccgc

SEQ ID NO: 120 - DNA, 91 bp, synthetic construct
  source 1..91: mol_type = other DNA
catgtggatctgaaacatgtggatctgaaacatgtggatctgaaacatgtggatctgaaatggccgcgcgcgcgcgcgcgcgcgcgcgcgc

SEQ ID NO: 121 - DNA, 75 bp, synthetic construct
  source 1..75: mol_type = other DNA
cgcgtgctgaaacaggcgcgcgtgctgaaacaggcgcgcgtgctgaaacaggcgcgcgtgctgaaacagcattgg

SEQ ID NO: 122 - DNA, 102 bp, synthetic construct
  source 1..102: mol_type = other DNA
cgcgtgctgaaacaggcgcgcgtgctgaaacaggcgcgcgtgctgaaacaggcgcgcgtgctgaaacagcattggcgccgccgccgccgccgccgccgccgc

SEQ ID NO: 123 - DNA, 102 bp, synthetic construct
  source 1..102: mol_type = other DNA
cgcgtgctgaaacaggcgcgcgtgctgaaacaggcgcgcgtgctgaaacaggcgcgcgtgctgaaacagcattggcgcgcgcgcgcgcgcgcgcgcgcgcgc

SEQ ID NO: 124 - DNA, 846 bp, synthetic construct
  source 1..846: mol_type = other DNA
gtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 125 - DNA, 540 bp, synthetic construct
  source 1..540: mol_type = other DNA
gtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaac

SEQ ID NO: 126 - DNA, 78 bp, synthetic construct
  source 1..78: mol_type = other DNA
catatggcggtgctgaaaacccgccgcagcacccgccgcagcacccgccgcgcggtgctgaaagattgggaacatatg

SEQ ID NO: 127 - DNA, 96 bp, synthetic construct
  source 1..96: mol_type = other DNA
atggtggaagatccgcatcgcctgaaacgcctgaaacgcctggtggcgaacgcgcgcgcgcgcgcgcgcgcgcgcctggtgaacgatattgaaaac

SEQ ID NO: 128 - DNA, 126 bp, synthetic construct
  source 1..126: mol_type = other DNA
ggcagcaaagaaagcggcagcgtgagcagcgaacagctggcgcagtttcgcagcctggatgcgggcagcgcgagcgaactgaaccattttgaactgctgtataaaaccaacaaacagaaacaggtg

SEQ ID NO: 129 - DNA, 42 bp, synthetic construct
  source 1..42: mol_type = other DNA
gcgctggtgaccgcgctggtgaccgcgctggtgaccaccagc

SEQ ID NO: 130 - DNA, 426 bp, synthetic construct
  source 1..426: mol_type = other DNA
ggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgc

SEQ ID NO: 131 - DNA, 1539 bp, synthetic construct
  source 1..1539: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccggcgaaactggtgaaaaaactgctgaccaaatggctggcg

SEQ ID NO: 132 - DNA, 1566 bp, synthetic construct
  source 1..1566: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccggcgaaactggtgaaaaaactgctgaccaaatggctggcgcgccgccgccgccgccgccgccgccgc

SEQ ID NO: 133 - DNA, 1566 bp, synthetic construct
  source 1..1566: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccggcgaaactggtgaaaaaactgctgaccaaatggctggcgcgcgcgcgcgcgcgcgcgcgcgcgcgc

SEQ ID NO: 134 - DNA, 1527 bp, synthetic construct
  source 1..1527: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccagcagcgcaaacgcaaatggagcattctggcgccgctgggcaccaccctg

SEQ ID NO: 135 - DNA, 1553 bp, synthetic construct
  source 1..1553: mol_type = other DNA
catatggcgggctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccagcagcgcaaacgcaaatggagcattctggcgccgctgggcaccaccctgcgccgccgccgccgccgccgccgccgc

SEQ ID NO: 136 - DNA, 1554 bp, synthetic construct
  source 1..1554: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccagcagcgcaaacgcaaatggagcattctggcgccgctgggcaccaccctgcgcgcgcgcgcgcgcgcgcgcgcgcgc

SEQ ID NO: 137 - DNA, 1527 bp, synthetic construct
  source 1..1527: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgaccaccggcctgccggcgctgattagctggaaacgcaaacgccagcag

SEQ ID NO: 138 - DNA, 1554 bp, synthetic construct
  source 1..1554: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgaccaccggcctgccggcgctgattagctggaaacgcaaacgccagcagcgccgccgccgccgccgccgccgccgc

SEQ ID NO: 139 - DNA, 1554 bp, synthetic construct
  source 1..1554: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgaccaccggcctgccggcgctgattagctggaaacgcaaacgccagcagcgcgcgcgcgcgcgcgcgcgcgcgcgc

SEQ ID NO: 140 - DNA, 1551 bp, synthetic construct
  source 1..1551: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcag

SEQ ID NO: 141 - DNA, 1578 bp, synthetic construct
  source 1..1578: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagcgccgccgccgccgccgccgccgccgc

SEQ ID NO: 142 - DNA, 1578 bp, synthetic construct
  source 1..1578: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagcgcgcgcgcgcgcgcgcgcgcgcgcgc

SEQ ID NO: 143 - DNA, 1552 bp, synthetic construct
  source 1..1552: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagc

SEQ ID NO: 144 - DNA, 1579 bp, synthetic construct
  source 1..1579: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagccgccgccgccgccgccgccgccgccgc

SEQ ID NO: 145 - DNA, 1579 bp, synthetic construct
  source 1..1579: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagccgcgcgcgcgcgcgcgcgcgcgcgcgc

SEQ ID NO: 146 - DNA, 1557 bp, synthetic construct
  source 1..1557: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgattaaaaaagcgctggcggcgctggcgaaactgaacattaaaggcctgctgtatggcgcgagcaacctgacctggggc

SEQ ID NO: 147 - DNA, 1585 bp, synthetic construct
  source 1..1585: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgattaaaaaagcgctggcggcgctggcgaaactgaacattaaaggcctgctgtatggcgcgagcaacctgacctggggcccgccgccgccgccgccgccgccgccgc

SEQ ID NO: 148 - DNA, 1585 bp, synthetic construct
  source 1..1585: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgattaaaaaagcgctggcggcgctggcgaaactgaacattaaaggcctgctgtatggcgcgagcaacctgacctggggcccgcgcgcgcgcgcgcgcgcgcgcgcgc

SEQ ID NO: 149 - DNA, 1539 bp, synthetic construct
  source 1..1539: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccatgtggatctgaaacatgtggatctgaaacatgtggatctgaaacatgtggatctgaaatgg

SEQ ID NO: 150 - DNA, 1567 bp, synthetic construct
  source 1..1567: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccatgtggatctgaaacatgtggatctgaaacatgtggatctgaaacatgtggatctgaaatggccgccgccgccgccgccgccgccgccgc

SEQ ID NO: 151 - DNA, 1567 bp, synthetic construct
  source 1..1567: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccatgtggatctgaaacatgtggatctgaaacatgtggatctgaaacatgtggatctgaaatggccgcgcgcgcgcgcgcgcgcgcgcgcgc

SEQ ID NO: 152 - DNA, 1551 bp, synthetic construct
  source 1..1551: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccgcgtgctgaaacaggcgcgcgtgctgaaacaggcgcgcgtgctgaaacaggcgcgcgtgctgaaacagcattgg

SEQ ID NO: 153 - DNA, 1578 bp, synthetic construct
  source 1..1578: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccgcgtgctgaaacaggcgcgcgtgctgaaacaggcgcgcgtgctgaaacaggcgcgcgtgctgaaacagcattggcgccgccgccgccgccgccgccgccgc

SEQ ID NO: 154 - DNA, 1578 bp, synthetic construct
  source 1..1578: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccgcgtgctgaaacaggcgcgcgtgctgaaacaggcgcgcgtgctgaaacaggcgcgcgtgctgaaacagcattggcgcgcgcgcgcgcgcgcgcgcgcgcgc

SEQ ID NO: 155 - DNA, 1233 bp, synthetic construct
  source 1..1233: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccggcgaaactggtgaaaaaactgctgaccaaatggctggcg

SEQ ID NO: 156 - DNA, 1260 bp, synthetic construct
  source 1..1260: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccggcgaaactggtgaaaaaactgctgaccaaatggctggcgcgccgccgccgccgccgccgccgccgc

SEQ ID NO: 157 - DNA, 1260 bp, synthetic construct
  source 1..1260: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccggcgaaactggtgaaaaaactgctgaccaaatggctggcgcgcgcgcgcgcgcgcgcgcgcgcgcgc

SEQ ID NO: 158 - DNA, 1221 bp, synthetic construct
  source 1..1221: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccagcagcgcaaacgcaaatggagcattctggcgccgctgggcaccaccctg

SEQ ID NO: 159 - DNA, 1248 bp, synthetic construct
  source 1..1248: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccagcagcgcaaacgcaaatggagcattctggcgccgctgggcaccaccctgcgccgccgccgccgccgccgccgccgc

SEQ ID NO: 160 - DNA, 1248 bp, synthetic construct
  source 1..1248: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccagcagcgcaaacgcaaatggagcattctggcgccgctgggcaccaccctgcgcgcgcgcgcgcgcgcgcgcgcgcgc

SEQ ID NO: 161 - DNA, 1221 bp, synthetic construct
  source 1..1221: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgaccaccggcctgccggcgctgattagctggaaacgcaaacgccagcag

SEQ ID NO: 162 - DNA, 1248 bp, synthetic construct
  source 1..1248: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgaccaccggcctgccggcgctgattagctggaaacgcaaacgccagcagcgccgccgccgccgccgccgccgccgc

SEQ ID NO: 163 - DNA, 1248 bp, synthetic construct
  source 1..1248: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgaccaccggcctgccggcgctgattagctggaaacgcaaacgccagcagcgcgcgcgcgcgcgcgcgcgcgcgcgc

SEQ ID NO: 164 - DNA, 1245 bp, synthetic construct
  source 1..1245: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcag

SEQ ID NO: 165 - DNA, 1272 bp, synthetic construct
  source 1..1272: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagcgccgccgccgccgccgccgccgccgc

SEQ ID NO: 166 - DNA, 1272 bp, synthetic construct
  source 1..1272: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagcgcgcgcgcgcgcgcgcgcgcgcgcgc

SEQ ID NO: 167 - DNA, 1246 bp, synthetic construct
  source 1..1246: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagc

SEQ ID NO: 168 - DNA, 1273 bp, synthetic construct
  source 1..1273: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagccgccgccgccgccgccgccgccgccgc

SEQ ID NO: 169 - DNA, 1273 bp, synthetic construct
  source 1..1273: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgctgcgtgaaacgcaaaaaaaaaccgctgaccaccggcctgccggcgctgattagctggaaacgcaaacgccagcagccgcgcgcgcgcgcgcgcgcgcgcgcgc

SEQ ID NO: 170 - DNA, 1251 bp, synthetic construct
  source 1..1251: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgattaaaaaagcgctggcggcgctggcgaaactgaacattaaaggcctgctgtatggcgcgagcaacctgacctggggc

SEQ ID NO: 171 - DNA, 1279 bp, synthetic construct
  source 1..1279: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgattaaaaaagcgctggcggcgctggcgaaactgaacattaaaggcctgctgtatggcgcgagcaacctgacctggggcccgccgccgccgccgccgccgccgccgc

SEQ ID NO: 172 - DNA, 1279 bp, synthetic construct
  source 1..1279: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgcctgattaaaaaagcgctggcggcgctggcgaaactgaacattaaaggcctgctgtatggcgcgagcaacctgacctggggcccgcgcgcgcgcgcgcgcgcgcgcgcgc

SEQ ID NO: 173 - DNA, 1233 bp, synthetic construct
  source 1..1233: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccatgtggatctgaaacatgtggatctgaaacatgtggatctgaaacatgtggatctgaaatgg

SEQ ID NO: 174 - DNA, 1261 bp, synthetic construct
  source 1..1261: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccatgtggatctgaaacatgtggatctgaaacatgtggatctgaaacatgtggatctgaaatggccgccgccgccgccgccgccgccgccgc

SEQ ID NO: 175 - DNA, 1261 bp, synthetic construct
  source 1..1261: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccatgtggatctgaaacatgtggatctgaaacatgtggatctgaaacatgtggatctgaaatggccgcgcgcgcgcgcgcgcgcgcgcgcgc

SEQ ID NO: 176 - DNA, 1245 bp, synthetic construct
  source 1..1245: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccgcgtgctgaaacaggcgcgcgtgctgaaacaggcgcgcgtgctgaaacaggcgcgcgtgctgaaacagcattgg

SEQ ID NO: 177 - DNA, 1272 bp, synthetic construct
  source 1..1272: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccgcgtgctgaaacaggcgcgcgtgctgaaacaggcgcgcgtgctgaaacaggcgcgcgtgctgaaacagcattggcgccgccgccgccgccgccgccgccgc

SEQ ID NO: 178 - DNA, 1272 bp, synthetic construct
  source 1..1272: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacggcagcaaagaaagcggcagcgtgagcagcgaacagctggcgcagtttcgcagcctggatgcgggcagcgcgagcgaactgaaccattttgaactgctgtataaaaccaacaaacagaaacaggtgggcgccgaagaatattgctataacattagcggccgcccggtgattggcgatccgaccgaagtgtatccggataacattagcaaagtgccgcatgataaaggccgcggcaacattggcaccagcattcgcaacctgccgcgccgcggcaactgccgcgtgtgctatagcggccagtatattggcatttatggcccggtgttttatcaggatgtggcgaacgcggaactgccgcgcatgctggtggaacataaaaccaaaatttgccatgtggtgaaacgcattggccgcgtgaccggcagcctgtatcatgtgctgaaatgcattaacggctgcaacgtgattaaatatagccgctgcggcctggaaccgagcagcctgaaatggattcgcaactttaccgattgcccgctgtgggtgaccagctgccgcgtgctgaaacaggcgcgcgtgctgaaacaggcgcgcgtgctgaaacaggcgcgcgtgctgaaacagcattggcgcgcgcgcgcgcgcgcgcgcgcgcgc

SEQ ID NO: 179 - DNA, 723 bp, synthetic construct
  source 1..723: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccggcgaaactggtgaaaaaactgctgaccaaatggctggcg

SEQ ID NO: 180 - DNA, 750 bp, synthetic construct
  source 1..750: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccggcgaaactggtgaaaaaactgctgaccaaatggctggcgcgccgccgccgccgccgccgccgccgc

SEQ ID NO: 181 - DNA, 750 bp, synthetic construct
  source 1..750: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccggcgaaactggtgaaaaaactgctgaccaaatggctggcgcgcgcgcgcgcgcgcgcgcgcgcgcgc

SEQ ID NO: 182 - DNA, 711 bp, synthetic construct
  source 1..711: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccagcagcgcaaacgcaaatggagcattctggcgccgctgggcaccaccctg

SEQ ID NO: 183 - DNA, 738 bp, synthetic construct
  source 1..738: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccagcagcgcaaacgcaaatggagcattctggcgccgctgggcaccaccctgcgccgccgccgccgccgccgccgccgc

SEQ ID NO: 184 - DNA, 738 bp, synthetic construct
  source 1..738: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccagcagcgcaaacgcaaatggagcattctggcgccgctgggcaccaccctgcgcgcgcgcgcgcgcgcgcgcgcgcgc

SEQ ID NO: 185 - DNA, 711 bp, synthetic construct
  source 1..711: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagcctgaccaccggcctgccggcgctgattagctggaaacgcaaacgccagcag

SEQ ID NO: 186 - DNA, 738 bp, synthetic construct
  source 1..738: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagcctgaccaccggcctgccggcgctgattagctggaaacgcaaacgccagcagcgccgccgccgccgccgccgccgccgc

SEQ ID NO: 187 - DNA, 738 bp, synthetic construct
  source 1..738: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagcctgaccaccggcctgccggcgctgattagctggaaacgcaaacgccagcagcgcgcgcgcgcgcgcgcgcgcgcgcgc

SEQ ID NO: 188 - DNA, 735 bp, synthetic construct
  source 1..735: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccgctgaccaccggcctgccggcgctgattagctggaaacgcaaacgccagcag

SEQ ID NO: 189 - DNA, 762 bp, synthetic construct
  source 1..762: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccgctgaccaccggcctgccggcgctgattagctggaaacgcaaacgccagcagcgccgccgccgccgccgccgccgccgc

SEQ ID NO: 190 - DNA, 762 bp, synthetic construct
  source 1..762: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccgctgaccaccggcctgccggcgctgattagctggaaacgcaaacgccagcagcgcgcgcgcgcgcgcgcgcgcgcgcgc

SEQ ID NO: 191 - DNA, 736 bp, synthetic construct
  source 1..736: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccgctgaccaccggcctgccggcgctgattagctggaaacgcaaacgccagcagc

SEQ ID NO: 192 - DNA, 763 bp, synthetic construct
  source 1..763: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccgctgaccaccggcctgccggcgctgattagctggaaacgcaaacgccagcagccgccgccgccgccgccgccgccgccgc

SEQ ID NO: 193 - DNA, 763 bp, synthetic construct
  source 1..763: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagctgcgtgaaacgcaaaaaaaaaccgctgaccaccggcctgccggcgctgattagctggaaacgcaaacgccagcagccgcgcgcgcgcgcgcgcgcgcgcgcgc

SEQ ID NO: 194 - DNA, 741 bp, synthetic construct
  source 1..741: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagcctgattaaaaaagcgctggcggcgctggcgaaactgaacattaaaggcctgctgtatggcgcgagcaacctgacctggggc

SEQ ID NO: 195 - DNA, 769 bp, synthetic construct
  source 1..769: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagcctgattaaaaaagcgctggcggcgctggcgaaactgaacattaaaggcctgctgtatggcgcgagcaacctgacctggggcccgccgccgccgccgccgccgccgccgc

SEQ ID NO: 196 - DNA, 769 bp, synthetic construct
  source 1..769: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagcctgattaaaaaagcgctggcggcgctggcgaaactgaacattaaaggcctgctgtatggcgcgagcaacctgacctggggcccgcgcgcgcgcgcgcgcgcgcgcgcgc

SEQ ID NO: 197 - DNA, 723 bp, synthetic construct
  source 1..723: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccatgtggatctgaaacatgtggatctgaaacatgtggatctgaaacatgtggatctgaaatgg

SEQ ID NO: 198 - DNA, 751 bp, synthetic construct
  source 1..751: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccatgtggatctgaaacatgtggatctgaaacatgtggatctgaaacatgtggatctgaaatggccgccgccgccgccgccgccgccgccgc

SEQ ID NO: 199 - DNA, 751 bp, synthetic construct
  source 1..751: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccatgtggatctgaaacatgtggatctgaaacatgtggatctgaaacatgtggatctgaaatggccgcgcgcgcgcgcgcgcgcgcgcgcgc

SEQ ID NO: 200 - DNA, 735 bp, synthetic construct
  source 1..735: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccgcgtgctgaaacaggcgcgcgtgctgaaacaggcgcgcgtgctgaaacaggcgcgcgtgctgaaacagcattgg

SEQ ID NO: 201 - DNA, 762 bp, synthetic construct
  source 1..762: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccgcgtgctgaaacaggcgcgcgtgctgaaacaggcgcgcgtgctgaaacaggcgcgcgtgctgaaacagcattggcgccgccgccgccgccgccgccgccgc

SEQ ID NO: 202 - DNA, 762 bp, synthetic construct
  source 1..762: mol_type = other DNA
catatggcggtgctgaaaacccgccgcagcacccgccgcagcacccgccgcgcggtgctgaaagattgggaacatatggtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgtgtatgtgaaagtgccggatacctggaccggcattccgaacattcattattggaacaccaccggcggcaccaccacctggccgggcaaacagatgaaagaagaaggcaacgaaatttatagcattattattccgaaaagctttggcgatgtgagcattattattaacgatggcaacaacgcgctggtgaccgcgctggtgaccgcgctggtgaccaccagccgcgtgctgaaacaggcgcgcgtgctgaaacaggcgcgcgtgctgaaacaggcgcgcgtgctgaaacagcattggcgcgcgcgcgcgcgcgcgcgcgcgcgc

SEQ ID NO: 203 - DNA, 1029 bp, synthetic construct
  source 1..1029: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccggcgaaactggtgaaaaaactgctgaccaaatggctggcg

SEQ ID NO: 204 - DNA, 1056 bp, synthetic construct
  source 1..1056: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccggcgaaactggtgaaaaaactgctgaccaaatggctggcgcgccgccgccgccgccgccgccgccgc

SEQ ID NO: 205 - DNA, 1056 bp, synthetic construct
  source 1..1056: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccggcgaaactggtgaaaaaactgctgaccaaatggctggcgcgcgcgcgcgcgcgcgcgcgcgcgcgc

SEQ ID NO: 206 - DNA, 1017 bp, synthetic construct
  source 1..1017: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagccagcagcgcaaacgcaaatggagcattctggcgccgctgggcaccaccctg

SEQ ID NO: 207 - DNA, 1044 bp, synthetic construct
  source 1..1044: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagccagcagcgcaaacgcaaatggagcattctggcgccgctgggcaccaccctgcgccgccgccgccgccgccgccgccgc

SEQ ID NO: 208 - DNA, 1044 bp, synthetic construct
  source 1..1044: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagccagcagcgcaaacgcaaatggagcattctggcgccgctgggcaccaccctgcgcgcgcgcgcgcgcgcgcgcgcgcgc

SEQ ID NO: 209 - DNA, 1017 bp, synthetic construct
  source 1..1017: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcctgaccaccggcctgccggcgctgattagctggaaacgcaaacgccagcag

SEQ ID NO: 210 - DNA, 1044 bp, synthetic construct
  source 1..1044: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcctgaccaccggcctgccggcgctgattagctggaaacgcaaacgccagcagcgccgccgccgccgccgccgccgccgc

SEQ ID NO: 211 - DNA, 1044 bp, synthetic construct
  source 1..1044: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcctgaccaccggcctgccggcgctgattagctggaaacgcaaacgccagcagcgcgcgcgcgcgcgcgcgcgcgcgcgc

SEQ ID NO: 212 - DNA, 1041 bp, synthetic construct
  source 1..1041: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccgctgaccaccggcctgccggcgctgattagctggaaacgcaaacgccagcag

SEQ ID NO: 213 - DNA, 1068 bp, synthetic construct
  source 1..1068: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccgctgaccaccggcctgccggcgctgattagctggaaacgcaaacgccagcagcgccgccgccgccgccgccgccgccgc

SEQ ID NO: 214 - DNA, 1068 bp, synthetic construct
  source 1..1068: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccgctgaccaccggcctgccggcgctgattagctggaaacgcaaacgccagcagcgcgcgcgcgcgcgcgcgcgcgcgcgc

SEQ ID NO: 215 - DNA, 1042 bp, synthetic construct
  source 1..1042: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccgctgaccaccggcctgccggcgctgattagctggaaacgcaaacgccagcagc

SEQ ID NO: 216 - DNA, 1069 bp, synthetic construct
  source 1..1069: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccgctgaccaccggcctgccggcgctgattagctggaaacgcaaacgccagcagccgccgccgccgccgccgccgccgccgc

SEQ ID NO: 217 - DNA, 1068 bp, synthetic construct
  source 1..1068: mol_type = other DNA
catatggcggtgctgaaaacccgccgcagcacccgccgcagcacccgccgcgcggtgctaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagctgcgtgaaacgcaaaaaaaaaccgctgaccaccggcctgccggcgctgattagctggaaacgcaaacgccagcagccgcgcgcgcgcgcgcgcgcgcgcgcgc

SEQ ID NO: 218 - DNA, 1047 bp, synthetic construct
  source 1..1047: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcctgattaaaaaagcgctggcggcgctggcgaaactgaacattaaaggcctgctgtatggcgcgagcaacctgacctggggc

SEQ ID NO: 219 - DNA, 1075 bp, synthetic construct
  source 1..1075: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcctgattaaaaaagcgctggcggcgctggcgaaactgaacattaaaggcctgctgtatggcgcgagcaacctgacctggggcccgccgccgccgccgccgccgccgccgc

SEQ ID NO: 220 - DNA, 1075 bp, synthetic construct
  source 1..1075: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcctgattaaaaaagcgctggcggcgctggcgaaactgaacattaaaggcctgctgtatggcgcgagcaacctgacctggggcccgcgcgcgcgcgcgcgcgcgcgcgcgc

SEQ ID NO: 221 - DNA, 1029 bp, synthetic construct
  source 1..1029: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagccatgtggatctgaaacatgtggatctgaaacatgtggatctgaaacatgtggatctgaaatgg

SEQ ID NO: 222 - DNA, 1057 bp, synthetic construct
  source 1..1057: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagccatgtggatctgaaacatgtggatctgaaacatgtggatctgaaacatgtggatctgaaatggccgccgccgccgccgccgccgccgccgc

SEQ ID NO: 223 - DNA, 1057 bp, synthetic construct
  source 1..1057: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagccatgtggatctgaaacatgtggatctgaaacatgtggatctgaaacatgtggatctgaaatggccgcgcgcgcgcgcgcgcgcgcgcgcgc

SEQ ID NO: 224 - DNA, 1042 bp, synthetic construct
  source 1..1042: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcccgcgtgctgaaacaggcgcgcgtgctgaaacaggcgcgcgtgctgaaacaggcgcgcgtgctgaaacagcattgg

SEQ ID NO: 225 - DNA, 1069 bp, synthetic construct
  source 1..1069: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcccgcgtgctgaaacaggcgcgcgtgctgaaacaggcgcgcgtgctgaaacaggcgcgcgtgctgaaacagcattggcgccgccgccgccgccgccgccgccgc

SEQ ID NO: 226 - DNA, 1069 bp, synthetic construct
  source 1..1069: mol_type = other DNA
catatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagtgcgctggtgaccgcgctggtgaccgcgctggtgaccaccagcccgcgtgctgaaacaggcgcgcgtgctgaaacaggcgcgcgtgctgaaacaggcgcgcgtgctgaaacagcattggcgcgcgcgcgcgcgcgcgcgcgcgcgc

SEQ ID NO: 227 - DNA, 987 bp, synthetic construct
  source 1..987: mol_type = other DNA
tgcgtgaaacgcaaaaaaaaaccggcgaaactggtgaaaaaactgctgaccaaatggctggcgcatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 228 - DNA, 1014 bp, synthetic construct
  source 1..1014: mol_type = other DNA
tgcgtgaaacgcaaaaaaaaaccggcgaaactggtgaaaaaactgctgaccaaatggctggcgcgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 229 - DNA, 1014 bp, synthetic construct
  source 1..1014: mol_type = other DNA
tgcgtgaaacgcaaaaaaaaaccggcgaaactggtgaaaaaactgctgaccaaatggctggcgcgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 230 - DNA, 975 bp, synthetic construct
  source 1..975: mol_type = other DNA
cagcagcgcaaacgcaaatggagcattctggcgccgctgggcaccaccctgcatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 231 - DNA, 1002 bp, synthetic construct
  source 1..1002: mol_type = other DNA
cagcagcgcaaacgcaaatggagcattctggcgccgctgggcaccaccctgcgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 232 - DNA, 1002 bp, synthetic construct
  source 1..1002: mol_type = other DNA
cagcagcgcaaacgcaaatggagcattctggcgccgctgggcaccaccctgcgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 233 - DNA, 975 bp, synthetic construct
  source 1..975: mol_type = other DNA
ctgaccaccggcctgccggcgctgattagctggaaacgcaaacgccagcagcatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 234 - DNA, 1002 bp, synthetic construct
  source 1..1002: mol_type = other DNA
ctgaccaccggcctgccggcgctgattagctggaaacgcaaacgccagcagcgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 235 - DNA, 1002 bp, synthetic construct
  source 1..1002: mol_type = other DNA
ctgaccaccggcctgccggcgctgattagctggaaacgcaaacgccagcagcgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 236 - DNA, 999 bp, synthetic construct
  source 1..999: mol_type = other DNA
tgcgtgaaacgcaaaaaaaaaccgctgaccaccggcctgccggcgctgattagctggaaacgcaaacgccagcagcatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 237 - DNA, 1026 bp, synthetic construct
  source 1..1026: mol_type = other DNA
tgcgtgaaacgcaaaaaaaaaccgctgaccaccggcctgccggcgctgattagctggaaacgcaaacgccagcagcgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 238 - DNA, 1026 bp, synthetic construct
  source 1..1026: mol_type = other DNA
tgcgtgaaacgcaaaaaaaaaccgctgaccaccggcctgccggcgctgattagctggaaacgcaaacgccagcagcgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 239 - DNA, 1000 bp, synthetic construct
  source 1..1000: mol_type = other DNA
tgcgtgaaacgcaaaaaaaaaccgctgaccaccggcctgccggcgctgattagctggaaacgcaaacgccagcagccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 240 - DNA, 1027 bp, synthetic construct
  source 1..1027: mol_type = other DNA
tgcgtgaaacgcaaaaaaaaaccgctgaccaccggcctgccggcgctgattagctggaaacgcaaacgccagcagccgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 241 - DNA, 1027 bp, synthetic construct
  source 1..1027: mol_type = other DNA
tgcgtgaaacgcaaaaaaaaaccgctgaccaccggcctgccggcgctgattagctggaaacgcaaacgccagcagccgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 242 - DNA, 1004 bp, synthetic construct
  source 1..1004: mol_type = other DNA
ctgattaaaaaagcgctggcggcgctggcgaaactgaacattaaaggcctgctgtatggcgcgagcaacctgacctggggccatatggcggtgctgaaaacccgccgcagcacccgccgcagcacccgccgcgcggtgctgaaagttgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 243 - DNA, 1033 bp, synthetic construct
  source 1..1033: mol_type = other DNA
ctgattaaaaaagcgctggcggcgctggcgaaactgaacattaaaggcctgctgtatggcgcgagcaacctgacctggggcccgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 244 - DNA, 1033 bp, synthetic construct
  source 1..1033: mol_type = other DNA
ctgattaaaaaagcgctggcggcgctggcgaaactgaacattaaaggcctgctgtatggcgcgagcaacctgacctggggcccgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 245 - DNA, 987 bp, synthetic construct
  source 1..987: mol_type = other DNA
catgtggatctgaaacatgtggatctgaaacatgtggatctgaaacatgtggatctgaaatggcatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 246 - DNA, 1015 bp, synthetic construct
  source 1..1015: mol_type = other DNA
catgtggatctgaaacatgtggatctgaaacatgtggatctgaaacatgtggatctgaaatggccgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 247 - DNA, 1015 bp, synthetic construct
  source 1..1015: mol_type = other DNA
catgtggatctgaaacatgtggatctgaaacatgtggatctgaaacatgtggatctgaaatggccgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 248 - DNA, 999 bp, synthetic construct
  source 1..999: mol_type = other DNA
cgcgtgctgaaacaggcgcgcgtgctgaaacaggcgcgcgtgctgaaacaggcgcgcgtgctgaaacagcattggcatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 249 - DNA, 1026 bp, synthetic construct
  source 1..1026: mol_type = other DNA
cgcgtgctgaaacaggcgcgcgtgctgaaacaggcgcgcgtgctgaaacaggcgcgcgtgctgaaacagcattggcgccgccgccgccgccgccgccgccgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 250 - DNA, 1026 bp, synthetic construct
  source 1..1026: mol_type = other DNA
cgcgtgctgaaacaggcgcgcgtgctgaaacaggcgcgcgtgctgaaacaggcgcgcgtgctgaaacagcattggcgcgcgcgcgcgcgcgcgcgcgcgcgccatatggcggtgctgaaaacccgccgcagcacccgccgcagcacccgccgcgcggtgctgaaagattgggaacatatggtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgtgtatgtgaaaccggtgtatgtgaaagtgtatgtgccgaacattcattattggaacaccaccggcaccaccaccggcaccacctggccgaccgcgatgaaagaagaaggcgaagaaatttatagcattattattccgagcagcagcggcgatgtgagcattattattccgaacattcattattggaacaccaccggcaccaccacctggccgaccgcgatgaaagaaatttatagcattattattccgagcagcagcggcgatgtgagcattattattccgaacattcattattggaacaccaccggcaccacctggccggcgatgaaagaagatggcgaaatttatagcattattattccgagcggcgatgtgagcattattattaacgatggcaacaacgcgctggtgaccgcgctggtgaccgcgctggtgaccactagt

SEQ ID NO: 251 - protein, 5 aa, synthetic construct
IHYWN

SEQ ID NO: 252 - protein, 5 aa, synthetic construct
VYVKV